(19) United States
(12) Patent Application Publication
(10) Pub. No.: US 2023/0144707 A1
(43) Pub. Date: May 11, 2023

(54) SMART BOTTLE AND ITS CONTROL METHOD

(71) Applicant: Littleone Inc., Seongnam-si (KR)

(72) Inventor: Byung Kyu LEE, Seoul (KR)

(73) Assignee: Littleone Inc., Seongnam-si (KR)

(21) Appl. No.: 17/983,411

(22) Filed: Nov. 9, 2022

(30) Foreign Application Priority Data
Nov. 9, 2021 (KR) 10-2021-0153176

(51) Int. Cl. A61J 9/00; G01G 17/04; C02F 1/32; A61J 11/00; A61L 2/10; A61L 2/24

(52) U.S. Cl. CPC A61J 9/00; G01G 17/04; C02F 1/325; A61J 11/008; A61L 2/10; A61L 2/24; C02F 2303/04; A61J 2200/74; A61J 2200/42; A61L 2202/23; A61L 2202/11; A61L 2202/14

(57) ABSTRACT

The present disclosure relates to a smart bottle and a method of controlling the same. The smart bottle includes a bottle for containing liquid and a base that is disposed under the bottle and includes an elastic body that is elastically deformed by the weight of the bottle and the base, a weight measurement sensor disposed above the elastic body and measuring the weight of the bottle by measuring a pressure that the elastic body transfers in a vertical direction from the ground as a reaction to gravity, and a controller that measures the amount of fed milk based on the change in the weight measured by the weight measurement sensor.

### CROSS-REFERENCE TO RELATED APPLICATIONS

This application claims the priority of Korean Patent Application No. 10-2021-0153176 filed on Nov. 9, 2021, in the Korean Intellectual Property Office, the disclosure of which is incorporated herein by reference.

### BACKGROUND OF THE DISCLOSURE

### Technical Field

The present disclosure relates to a smart bottle, and, more particularly, to a smart bottle capable of measuring the amount of fed milk, a feeding pattern, etc., and a method of controlling the same.

### Background Art

A bottle is a container used to contain liquid. Among them, a feeding bottle is generally used for bottle-feeding. However, the recommended amount of milk to be fed and the number of times of feeding for a baby vary depending on the baby's age in months, weight, etc., and, in relation to preparation for formula milk, an appropriate temperature of a bottle differs depending on the type of formula. Therefore, in general, a caregiver raising a child manually records the amount of fed milk, the number of times of feeding, etc. from memory. However, with the recent development of Internet of Things (IoT) technology, there has been an increasing demand for the development of more efficient methods of preparing formula milk and feeding in connection with childcare.

In this regard, Korean Patent Application Publication No. 10-2017-0057026 published on May 24, 2017 under the title of invention of “The Nursing Bottle Warmer and the Method of Nursing Management” describes a method of calculating the amount of fed milk by measuring the weight of a bottle and managing the records of bottle-feeding by calculating the starting time and the ending time of the feeding. However, in order to measure the amount of the fed milk and manage the records of the feeding by the method, it is necessary to mount the bottle on a bottle warmer. Therefore, it is not possible to measure the amount of the fed milk or manage the records of the feeding at a place where the bottle warmer is not provided. In addition, there is a problem in that the weight of a bottle cannot be accurately measured when a bottle warmer is not placed on a flat place while the amount of fed milk is measured so that it is difficult to accurately calculate the amount of fed milk.

Meanwhile, Korean Patent Application Publication No. 10-2017-0071999 published on Jun. 26, 2017 under the title of invention of “Smart Bottle” describes a smart bottle that includes a measuring unit for measuring weight under a body part of the bottle in which liquid is contained. However, since such an apparatus simply measures the weight of a bottle before and after drinking to measure the amount of fed milk, it is not possible to separately determine whether bottle-feeding is in progress. As a result, there is a problem in that a weight measurement sensor needs to be working in real time to determine whether the bottle-feeding is in progress.

Consequently, in the technical field, there is a demand for a method of measuring the amount of fed milk and a smart bottle using the same by which it is possible to determine whether bottle-feeding is in progress by a method other than measuring weight and measure the accurate amount of fed milk after the completion of the bottle-feeding.

### RELATED ART DOCUMENT

### Patent Document

(Patent Document 1) Korean Patent Application Publication No. 10-2017-0057026 published on May 24, 2017 under the title of invention of “The Nursing Bottle Warmer and the Method of Nursing Management”
(Patent Document 2) Korean Patent Application Publication No. 10-2017-0071999 published on Jun. 26, 2017 under the title of invention of “Smart Bottle”

### SUMMARY OF THE DISCLOSURE

The purpose of the present disclosure is to provide a method of measuring the amount of fed milk and a smart bottle using the same by which it is possible to determine whether bottle-feeding is in progress by a method other than measuring weight.

Another purpose of the present disclosure is to provide a method of measuring the amount of fed milk and a smart bottle using the same by which it is possible to accurately measure the amount of fed milk and efficiently record the measured amount of fed milk.

According to an embodiment of the present disclosure, there is provided a smart bottle including a bottle for containing liquid and a base that is disposed under the bottle, wherein the base includes an elastic body that is elastically deformed by the weight of the bottle and the base, a weight measurement sensor disposed above the elastic body and measuring the weight of the bottle by measuring a pressure that the elastic body transfers in a vertical direction from the ground as a reaction to gravity, and a controller that measures the amount of fed milk based on the change in the weight measured by the weight measurement sensor.

According to another embodiment of the present disclosure, the apparatus further includes a lid and the bottle includes a lid recognition sensor for determining whether the lid is engaged.

According to still another embodiment of the present disclosure, the apparatus determines that formula preparation for milk has started when the lid is not engaged while the bottle is empty and determines that the formula preparation for milk has ended when the lid is engaged while the bottle is filled with contents.

According to yet another embodiment of the present disclosure, the controller determines that bottle-feeding has started when the lid is not engaged while the bottle is filled with contents and determines that the bottle-feeding has ended when the weight of the bottle is reduced and the lid is engaged.

According to yet another embodiment of the present disclosure, the controller measures the amount of fed milk based on the change in the weight of the bottle.

According to yet another embodiment of the present disclosure, the base further includes an ultraviolet (UV-C) lamp for sterilizing the contents in the bottle by emitting ultraviolet light toward the bottle.

According to yet another embodiment of the present disclosure, the ultraviolet lamp emits UV light when the weight of the bottle is equal to or greater than a threshold value.

According to yet another embodiment of the present disclosure, the base further includes an inclination sensor for measuring the inclination of the bottle, and the ultraviolet lamp emits UV light when the inclination of the bottle is in an error of less than or equal to a threshold value with respect to a vertical direction of the ground.

According to yet another embodiment of the present disclosure, the inclination sensor includes at least one of a gyro sensor and an acceleration sensor, and the controller calculates the inclination of the bottle based on information on an angular velocity measured by the gyro sensor or information on an acceleration measured by the acceleration sensor.

According to yet another embodiment of the present disclosure, the weight measurement sensor includes at least one of a beam-type load cell, a three-wire load cell, and a columnar load cell.

According to yet another embodiment of the present disclosure, there is provided a method of operating an apparatus configured to be engageable on one side of a bottle, including the step of determining whether the bottle is empty by measuring the weight of liquid contained in the bottle, the step of determining whether a lid of the bottle is open, and the step of determining that preparation for formula milk has started when the lid is open while the bottle is empty.

According to yet another embodiment of the present disclosure, the method further includes the step of determining whether the bottle is being filled with liquid by measuring the weight of the bottle when it is determined that the preparation for formula milk has started, the step of determining whether the lid is closed, the step of determining that the preparation for formula milk has ended when the bottle lid is closed while the liquid is contained in the bottle, and the step of heating the bottle to a formulating temperature.

According to yet another embodiment of the present disclosure, the method further includes the step of determining whether liquid is contained in the bottle by measuring the weight of liquid contained in the bottle, the step of determining whether the bottle lid is open, and the step of determining that bottle-feeding has started when the bottle lid is open while the liquid is contained in the bottle.

According to a further embodiment of the present disclosure, the method further includes the step of determining whether the weight of the liquid in the bottle is decreasing by measuring the weight of the bottle when it is determined that the bottle-feeding has started, the step of determining whether the bottle lid is closed, the step of determining that the bottle-feeding has ended when the weight of the liquid is reduced and the bottle lid is closed, and the step of calculating the amount of fed milk and transmitting information on the calculated amount to a terminal.

According to the present disclosure, it is possible to determine whether bottle-feeding is in progress by a method other than measuring weight.

In addition, according to the present disclosure, it is possible to accurately measure the amount of fed milk and efficiently manage the records of the measured amount of fed milk.

### BRIEF DESCRIPTION OF THE DRAWINGS

FIG. 1 shows a system for managing bottle-feeding according to an embodiment of the present disclosure.

FIG. 2 shows a smart bottle according to an embodiment of the present disclosure.

FIG. 3 is a side view of a smart bottle according to another embodiment of the present disclosure.

FIG. 4 is a top plan view of a bottom surface of the smart bottle shown in FIG. 3 viewed from above.

FIG. 5 shows a structure of a base including a beam-type load cell.

FIG. 6 shows a structure of a base including a three-wire load cell.

FIG. 7 shows a structure of a base including a columnar load cell.

FIG. 8 shows a structure of the smart bottle according to an embodiment of the present disclosure.

FIG. 9 shows a method of determining whether formula preparation for milk has started or ended according to an embodiment of the present disclosure.

FIG. 10 shows a method of determining whether bottle-feeding has started or ended according to an embodiment of the present disclosure.

### DETAILED DESCRIPTION OF THE EMBODIMENT

The present disclosure will be described in detail with reference to the accompanying drawings as follows. Herein, repeated descriptions and detailed descriptions of well-known functions and features that may unnecessarily obscure the gist of the present disclosure will not be provided. The embodiments of the present disclosure are provided so that a person having ordinary skill in the art may more fully understand the present disclosure. Accordingly, the shapes, sizes, etc. of components shown in the drawings may be exaggerated for a clearer

### DESCRIPTION

Hereinafter, preferred embodiments according to the present disclosure will be described in detail with reference to the appended drawings.

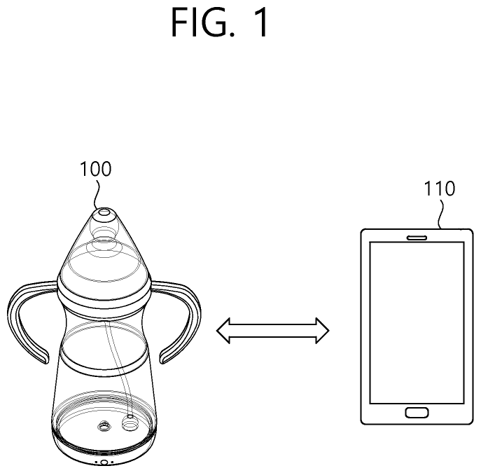

FIG. 1 shows a system for managing bottle-feeding according to an embodiment present disclosure.

Referring to FIG. 1, the system for managing bottle-feeding according to an embodiment of the present disclosure may include a smart bottle 100 and a portable terminal 110.

The smart bottle 100 may transmit information on bottle-feeding such as information on the temperature of the bottle, the inclination of the bottle, a water level in the bottle, the amount of fed milk, etc. to the portable terminal 110 based on a short-range communication. To this end, the smart bottle 100 may include a base device under the bottle. The base device may be integrally coupled to the bottle or may be detachably coupled to the bottle. The base device may be provided under the bottle as shown in FIG. 1 and may communicate with the portable terminal 110 based on a short-range communication standard such as Wireless Fidelity (WiFi) and Bluetooth.

The portable terminal 110 may refer to any device that a user can carry, and may be not only a small device such as a tablet PC, a smart phone, and a smart watch but also a device such as a laptop computer.

An application for exchanging information with the smart bottle 100 may be installed in the portable terminal 110. The application may obtain information on bottle-feeding from the smart bottle 100 based on the portable terminal 110, and may record it in a memory provided in the portable terminal 110. In addition, the application may generate information on a feeding pattern for a baby based on information on the baby's age in months received from a user through the portable terminal 110, the information on bottle-feeding received from the smart bottle 100, etc., and may display the information on bottle-feeding, the information on a feeding pattern, etc. recorded in the portable terminal 110 through a display of the portable terminal 110.

Furthermore, the application may acquire information on a recommended formulating temperature for powdered formula to be fed based on information on the powdered formula obtained by the portable terminal 110, and may control the portable terminal 110 to transmit the information on the recommended temperature to the smart bottle. In this case, the smart bottle 110 may set a formulating temperature based on the information on the recommended formulating temperature received from the portable terminal 110. The information on the powdered formula may be obtained from an image taken by a camera of the portable terminal 110, a universal product code (UPC), a European article number 13 (EAN13), a bar code, a quick response code (QR code), etc. Alternatively, a user may directly input the information on the powdered formula using the portable terminal 110.

Meanwhile, the application may generate information on a feeding pattern for a bay as shown in Table 1 below based on information on the baby's age in months received from a user through the portable terminal 110 and information on bottle-feeding received from the smart bottle 100.

TABLE 1

| Age | Number of times of | Feeding | |
|---|---|---|---|
| (month) | feeding per day | interval | Duration |
| Less than 1 | 10-12 | 1:30-2:00 | 0:10-0:40 |
| 1-2 | 10 | 2:00-2:30 | 0:10-0:30 |
| 3-4 | 8 | 2:30-3:00 | 0:10-0:30 |
| 5-6 | 6-7 | 3:00 | 0:10-0:30 |
| 7 or more | 6 | 3:30 | 0:10-0:30 |

The application may provide a user with an alarm to prepare for bottle-feeding by means of the portable terminal 110 based on information on a feeding pattern as shown in Table 1 when the next feeding time approaches. For example, in the case of a seven-month-old baby, when it is determined that feeding has ended at 12 o'clock based on information on bottle-feeding received from the smart bottle 100, the application may determine that the next feeding time will be 3:30 based on information on a feeding interval for seven or more-month-old babies, and may provide the alarm to prepare for bottle-feeding to the user based on a push alarm function, a vibration alarm function, a sound, etc. of the portable terminal 110 at 3:20, 10 minutes before the time. In addition, the application may compare information on a feeding pattern for the baby with information on feeding patterns for other babies of the same age as the baby for each region, age group and/or gender, and may display the comparison result by means of the portable terminal 110. Furthermore, the application may have a function that allows the user to set a formulating temperature or a feeding temperature, a function that allows the user to select a formulating mode or a feeding mode, and the like.

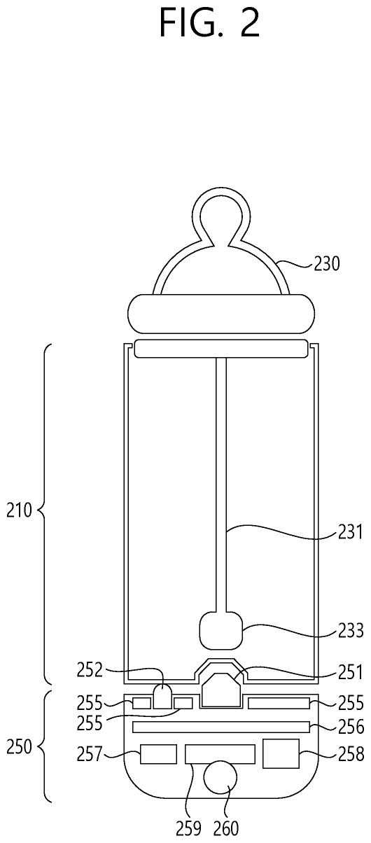

FIG. 2 shows the smart bottle according to an embodiment of the present disclosure.

Referring to FIG. 2, the smart bottle according to this embodiment may include a bottle 210, a nipple 230, and a base 250.

The bottle 210 may contain water, formula milk, milk, etc. The bottle 210 may be embodied with various materials such as glass and plastic as needed.

Here, the bottle 210 may be made of tempered glass that does not transmit ultraviolet (UV) light. Although not shown in FIG. 2, a water level sensor for measuring a water level in the bottle 210 may be provided in the bottle 210. The water level sensor may be a plurality of resistive sensors or at least one capacitance level sensor, for example.

The nipple 230 may be embodied in a replaceable form to be replaced based on a baby's age in months and the rate of feeding. A straw 231 may be coupled to one side of the nipple 230. In this case, the straw may include a weight 233 for preventing abdominal pains of the baby. The straw may be formed of a material having a predetermined elasticity so that the straw may remain in liquid in the bottle 210 by the weight even when the bottle 210 is tilted.

The base 250 may be configured to be integrally coupled to the bottle 210 or may be configured to be coupled to one side of the bottle. The base 250 may include a temperature sensor 251, an inclination sensor 252, a status indicator light 253, an ultraviolet lamp 254, a heater 255, a controller 256, a communication unit 257, a vibration motor 258, a battery 259, and a button 260.

The temperature sensor 251 may be positioned on a protrusion on an upper side of the base 250 so as to be spaced apart from the heater 255 by a predetermined distance. The heater 255 may be in a plate shape so that sufficient thermal energy may be transmitted to the bottle 210. When the temperature sensor 251 and the heater 255 are not spaced apart from each other by the predetermined distance, it may not possible that the temperature sensor 251 accurately measures the temperature of the bottle 210 due to the heat generated by the heater 255. Therefore, it may be desirable that the temperature sensor 251 is positioned on the protrusion on the upper side of the base 250, and, for this reason, a concave portion into which the temperature sensor 251 is inserted may be formed on a lower side of the bottle 210.

The inclination sensor 252 may obtain information on the inclination of the bottle. For example, the inclination sensor 252 may be at least one of a gyro sensor and an acceleration sensor. When the gyro sensor is used as the inclination sensor 252, the inclination may be calculated based on information on an angular velocity measured by the gyro sensor. The information on the inclination may be periodically transmitted to the portable terminal.

The heater 255 may heat liquid contained in the bottle 210. To this end, the heater 255 may have a plate shape as described above.

The controller 256 may include a processor or MCU and may perform all processes related to the smart bottle.

In this case, the processor or MCU may be mounted on a PCB substrate. In other words, the controller 256 may control the operation of the temperature sensor 251, the inclination sensor 252, the status indicator light 253, the ultraviolet lamp 254, the heater 255, the communication module 257, the vibration motor 258, the battery 259, etc. in the base 250.

The communication unit 257 may transmit information on bottle-feeding such as information on a temperature measured by the temperature sensor and information on an inclination measured by the inclination sensor, information on the remaining amount of the battery 259, etc. to an external device such as a repeater and a portable terminal.

In addition, the communication unit 257 may receive information on setting the feeding temperature, information on setting the formulating temperature, information on mode change, etc. from the external device.

The communication unit 257 may include an NFC chip, an NFC antenna, a Bluetooth module, a Wi-Fi module, a 5G communication module, and the like. For example, the communication unit 257 may communicate with the terminal by a Bluetooth 4.0 communication.

The vibration motor 258 may generate a vibration when raising an alarm is required under the control of the controller 256. For example, when the bottle 210 is tilted at an angle wider than a preset angle for a longer time than a preset time and the temperature of the bottle 210 measured by the temperature sensor 251 is higher than a preset temperature, a motor of the vibration motor 258 may be driven to enable a user to notice it.

The battery 259 may supply power required to drive the smart bottle. When the controller 256 checks the remaining amount of the battery 259 and determines that it is not possible to heat the bottle 210 to the formulating temperature and/or the feeding temperature, it may provide an alarm about the shortage of the remaining amount of the battery 259 by means of the status indicator light 253 and/or the vibration motor 258 without driving the heater 255. In that case as well, the portable terminal may also raise an alarm about the low remaining amount of the battery 259 by a push alarm, vibration, sound, etc.

The button 260 may be used to turn the base 250 on/off or to switch between the formulating mode and the feeding mode.

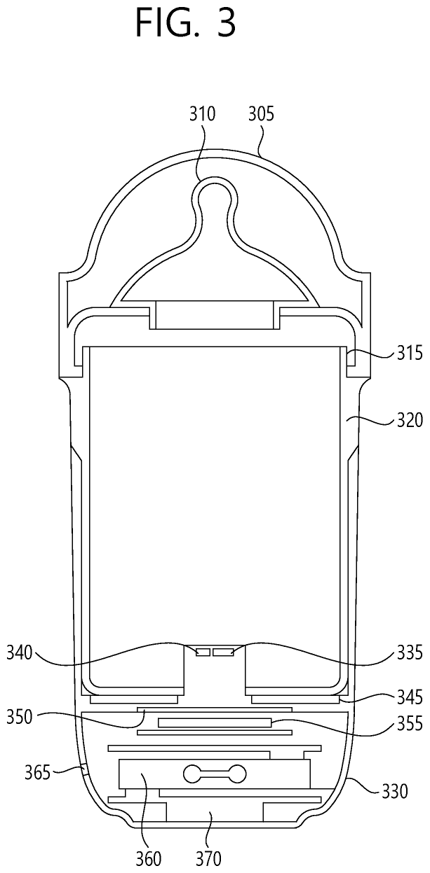

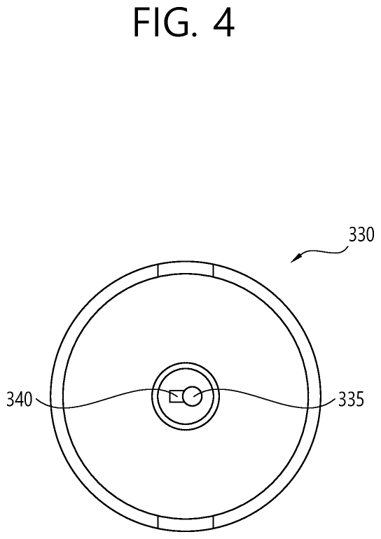

FIGS. 3 and 4 show a smart bottle according to another embodiment of the present disclosure. FIG. 3 is a side view of the smart bottle according to another embodiment of the present disclosure, and FIG. 4 is a top plan view of a bottom surface of the smart bottle shown in FIG. 3 viewed from above.

Referring to FIGS. 3 and 4, the smart bottle according to this embodiment may include a lid 305, a nipple 310, a feeding bottle coupling part 315, a bottle 320, and a base 330.

The lid 305 may be formed to cover the nipple to prevent contamination of the nipple. In addition, the lid 305 may be used to recognize the start and end of the formulating mode. For example, the feeding bottle coupling part 315 or the bottle 320 may include a sensor for determining whether the lid 305 has been coupled to determine whether the lid 305 has been mounted. A control device or controller consisting of a processor or MCU mounted on a substrate 350 may determine whether the formulating mode has started or ended and whether the feeding mode has started or ended based on whether the lid 305 has been attached and a change in the weight of the bottle. For example, when the lid 305 is not engaged while the bottle 320 is empty, it may be determined that the formulating mode has started, and, when the bottle 320 contains liquid and the lid 305 is mounted, it may be determined that the formulating mode has ended. Whether the bottle 320 is empty or contains liquid may be determined based on the measurement by a weight measurement sensor 360 in the base 330.

In addition, the lid 305 may be used to recognize the start and end of the feeding mode. For example, when the lid 305 is not engaged while the bottle 320 is filled with liquid, it may be determined that the feeding mode has started, and, when the weight of the liquid in the bottle 320 is reduced and the lid 305 is mounted back, it may be determined that the feeding mode has ended. In this case, the amount of fed milk may be calculated by calculating how much the weight of the liquid in the bottle 320 is reduced.

The nipple 310 may be embodied in a replaceable form to be replaced based on a baby's age in months and the rate of feeding.

The feeding bottle coupling part 315 may have a shape that enables the nipple 310 to be coupled to the bottle 320. For example, the feeding bottle coupling part 315 may be in a shape that enables the nipple 310 to be coupled to the bottle 320 by being turned.

The bottle 320 may contain water, formula milk, milk, etc. The bottle 320 may be formed of various materials such as glass and plastic as needed. In this case, the bottle 320 may be formed of PPSU and may be formed by injection molding.

Here, the bottle 320 may be formed of a tempered glass that does not transmit UV light. Although not shown in FIG. 3, the bottle 320 may include a water level sensor for measuring a water level in the bottle. The water level sensor may be a plurality of resistive sensors or at least one capacitance level sensor, for example.

The base 330 may be integrally coupled to the bottle 320 or may be configured to be coupled to one side of the bottle. The base 330 may include a temperature sensor 335, an ultraviolet lamp 340, a heater 345, the substrate 350, a battery 355, the weight measurement sensor 360, a status indicator light 365, and an elastic body 370.

The temperature sensor 335 may be positioned on a protrusion on an upper side of the base 330 so as to be spaced apart from the heater 345 by a predetermined distance. When the temperature sensor 335 and the heater 345 are not spaced apart from each other by the predetermined distance, it may not possible that the temperature sensor 335 accurately measures the temperature of the bottle 320 due to the heat generated by the heater 345. Therefore, it may be desirable that the temperature sensor 335 is positioned on the protrusion on the upper side of the base 330, and, for this reason, a concave portion into which the temperature sensor 335 is inserted may be formed on a lower side of the bottle 320.

The ultraviolet lamp 340 may emit UV light to sterilize the bottle 320 or liquid in the bottle 320. The ultraviolet lamp 340 may be a UV-C LED. In this case, the ultraviolet lamp 340 may emit light in a vertical direction so that the light may not be emitted to the outside of the bottle 320, and may emit the light only at an angle within a certain range.

Here, the ultraviolet lamp 340 may perform sterilization only when the bottle is filled with contents. In addition, the ultraviolet lamp 340 may emit UV light only when the weight of the bottle 320 measured by the weight measurement sensor 360 is equal to or greater than a threshold value.

On the other hand, the ultraviolet lamp 340 may emit UV light only when the bottle is placed in a vertical direction with respect to the ground. For example, although not shown in the drawings, the smart bottle may further include an inclination sensor, and the ultraviolet lamp 340 may emit UV light only when the inclination of the bottle is in error by less than or equal to a threshold value with respect to the vertical direction of the ground.

In addition, the ultraviolet lamp 340 may emit UV light only when it is determined that the lid 305 is coupled to the bottle 320 by a sensor for determining whether the lid 305 is coupled to the bottle 320 in order to protect a user of the smart bottle from UV light.

The heater 345 may have a plate shape so that sufficient thermal energy may be transmitted to the bottle 320. In this case, the heater 345 may be a film heater.

The substrate 350 may be equipped with a processor or an MCU and an electronic circuit necessary for driving the smart bottle.

The battery 355 may supply power required to drive the smart bottle.

The weight measurement sensor 360 may be disposed above the elastic body 370 and may measure the weight of the bottle 320 by measuring a pressure that the elastic body 370 transfers in a vertical direction from the ground as a reaction to gravity. In this case, the weight measurement sensor 360 may include a load cell such as a beam-type load cell, a columnar load cell, and a three-wire load cell.

The status indicator light 365 may indicate whether the temperature of the bottle 320 has reached the formulating temperature or the feeding temperature in the formulating mode or the feeding mode by a change in color. The status indicator light 365 may also indicate whether the remaining amount of the battery 355 is low. Here, the status indicator light 365 may be an LED.

The elastic body 370 may be elastically deformed by the weight of the lid 305, the nipple 310, the bottle 320, the base 330, etc. thereon. In this case, the elastic body 370 may be formed of silicon or a spring.

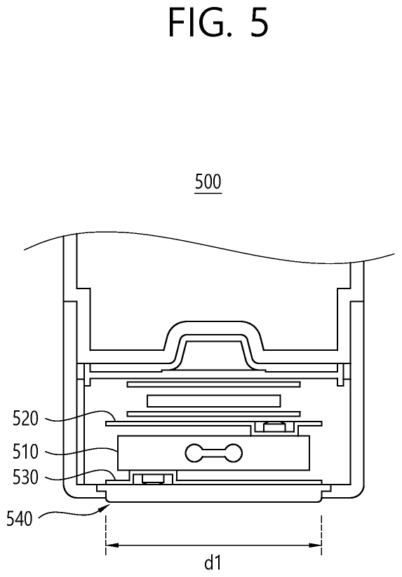

FIGS. 5 to 7 show examples of a base including a load cell according to various embodiments of the present disclosure.

FIG. 5 shows an embodiment of a base 500 including the beam-type load cell, and the base 500 may include a load cell 510, first and second fixing plates 520 and 530, and an elastic body 540.

The load cell 510 may be the beam-type load cell, and may measure the weight of the bottle by measuring a pressure that the elastic body 540 transfers in a vertical direction from the ground as a reaction to gravity.

The first and second fixing plates 520 and 530 may fix the load cell 510 so that the load cell 510 does not get out of the position therebetween.

The elastic body 540 may be elastically deformed by the weight of the components disposed thereon such as the bottle and the base. In this case, the area of the elastic body 540 may be the same as the bottom area of the first and second fixing plates 520 and 530 as shown in FIG. 5. Furthermore, the first and second fixing plates 520 and 530 and the elastic body 540 may all be circular and may have the same diameter d1.

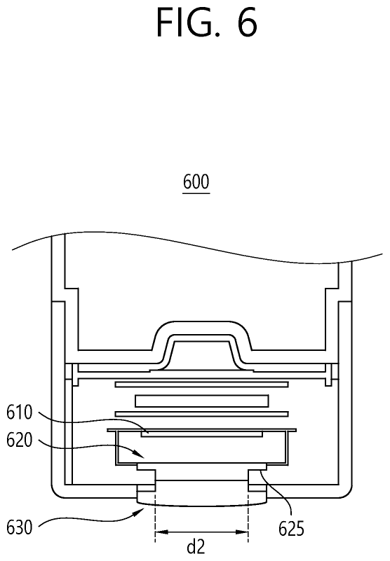

FIG. 6 shows an embodiment of a base 600 including the three-wire load cell, and the base 600 may include a load cell 610, a load cell case 620, an elastic transmission part 625, and an elastic body 630.

The load cell 610 may be the three-wire load cell, and may measure the weight of the bottle by measuring a pressure that the elastic body 630 transfers in a vertical direction from the ground as a reaction to gravity.

The load cell case 620 may fix the load cell 610 so that the load cell 610 does not get out of the load cell case 620. In addition, the load cell case 620 may be in contact with the load cell 610 and the elastic transmission part 625 to transmit the force of the elastic transmission part 625 to the load cell 610.

The elastic transmission part 625 may be disposed between the load cell case 620 and the elastic body 630 to transmit the force of the elastic body 630 to the load cell case 620. The area of the surface of the elastic transmission part 625 in contact with the load cell case 620 may be wider than the area of the surface in contact with the elastic body 630.

The elastic body 630 may be elastically deformed by the weight of the components disposed thereon such as the bottle and the base. In this case, the area of the surface of the elastic body 630 in contact with the elastic transmission part 625 may be narrower than the area of the surface in contact with the ground. Furthermore, the contact surface between the elastic body 630 and the elastic transmission part 625 may be circular, and the diameter of the circular contact surface may be d2.

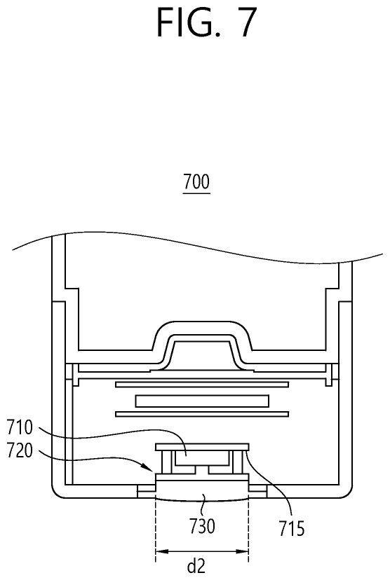

FIG. 7 shows an embodiment of a base 700 including the columnar load cell, and the base 700 may include a load cell 710, a load cell fixing part 715, an elastic transmission part 720, and an elastic body 730.

The load cell 710 may be the columnar load cell, and may measure the weight of the bottle by measuring a pressure that the elastic body 730 transfers in a vertical direction from the ground as a reaction to gravity.

The load cell fixing part 715 may fix the load cell 710 by covering all sides thereof so that the load cell 710 does not shake in a horizontal direction.

The elastic transmission part 720 may be disposed between the load cell 710 and the elastic body 730 to transmit the force of the elastic body 730 to the load cell 710. In order to direct pressure to the center of the load cell 710, the area of the surface of the elastic transmission part 720 in contact with the load cell 710 may be much narrower than the area of the surface in contact with the elastic body 730.

The elastic body 730 may be elastically deformed by the weight of the components disposed thereon such as the bottle and the base. In this case, the area of the surface of the elastic body 730 in contact with the elastic transmission part 720 may be narrower than the area of the surface in contact with the ground. Furthermore, the contact surface between the elastic body 730 and the elastic transmission part 720 may be circular, and the diameter of the circular contact surface may be d2. In this case, the diameter d2 may be longer than the diameter of the load cell 710.

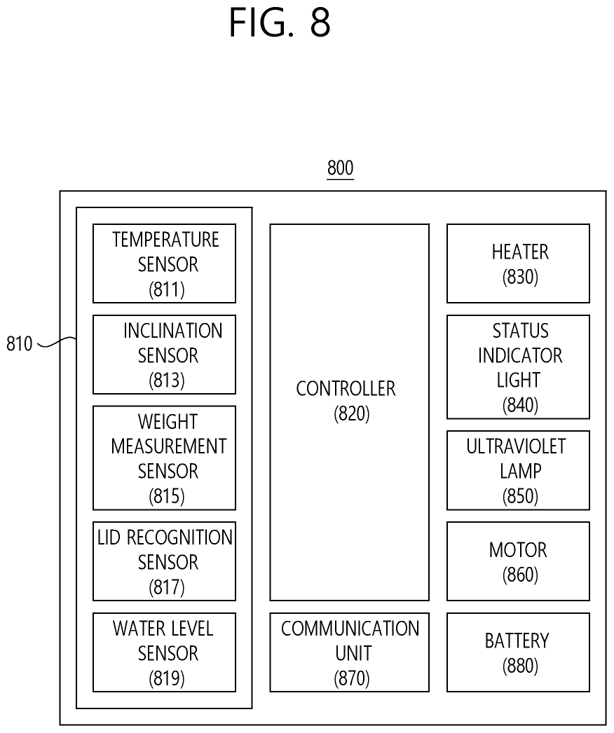

FIG. 8 shows a structure of a smart bottle 800 according to an embodiment of the present disclosure.

Referring to FIG. 8, the smart bottle 800 may include a sensor unit 810, a controller 820, a heater 830, a status indicator light 840, an ultraviolet lamp 850, a motor 860, a communication unit 870, and a battery 880.

The sensor unit 810 may include a temperature sensor 811, an inclination sensor 813, a weight measurement sensor 815, and a lid recognition sensor 817.

The temperature sensor 811 may acquire information on the temperature of a bottle.

The inclination sensor 813 may obtain information on the inclination of the bottle. For example, the inclination sensor 813 may be at least one of a gyro sensor and an acceleration sensor. When the gyro sensor is used as the inclination sensor 813, the inclination of the bottle may be calculated based on information on an angular velocity measured by the gyro sensor. When the acceleration sensor is used as the inclination sensor 813, the inclination of the bottle may be calculated based on information on an acceleration measured by the acceleration sensor. The information on the inclination may be periodically transmitted to a portal terminal.

The weight measurement sensor 815 may measure the total weight of the smart bottle or the weight of liquid contained in the bottle.

The lid recognition sensor 817 may determine whether the lid is coupled to the body. Accordingly, the lid recognition sensor 817 may be used to determine whether the formulating mode or the feeding mode has started or ended.

The controller 820 may perform all operations for controlling the sensor unit 810, the heater 830, the status indicator light 840, the ultraviolet lamp 850, the motor 860, the communication unit 870, and the battery 880.

The controller 820 may control the operation of the heater 830 based on information on temperature measured by the temperature sensor 811. The controller 820 may allow the information on temperature measured by the temperature sensor 811 to be periodically transmitted to the portable terminal. In addition, the controller 820 may determine that there is no liquid in the bottle when the temperature measured by the temperature sensor 811 rises faster than a preset speed, and may stop the operation of the heater 830 and provide an alarm by means of the status indicator light 840 and/or the motor 860. In this case, the portable terminal may also raise an alarm about the lack of liquid by a push alarm, vibration, sound, etc.

The controller 820 according to the present disclosure may operate in the formulating mode or the feeding mode.

The formulating mode may be defined as a mode in which the heater 830 is controlled to operate until the temperature of the bottle reaches the formulating temperature, and the feeding mode may be defined as a mode in which the heater 830 is controlled to maintain the feeding temperature. Here, the formulating temperature refers to a temperature suitable for preparing formula milk, and the feeding temperature refers to a temperature suitable for providing the milk to a baby. The formulating temperature may vary depending on the type, the manufacturer, etc. of powdered formula and may be generally higher than the feeding temperature. For example, when the smart bottle 800 is initially operated, the formulating temperature may be set to 40° C., and the feeding temperature may be set to 37° C. However, when information on a recommended formulating temperature is received from the portable terminal, the controller 820 may reset the formulating temperature accordingly. The information on the recommended formulating temperature may be obtained from information on powdered formula identified based on an image captured by a camera of the portable terminal, a UPC, an EAN13, a barcode, a QR code, etc.

When the temperature of the bottle initially reaches the formulating temperature as a result of the operation of the heater 830 in the formulating mode, the controller 820 may indicate that formula preparation for milk has been completed by means of the status indicator light 840 and/or the motor 860 provided in the smart bottle 800. In this case, the portal terminal may also provide a user with an alarm about the completion of formula preparation for milk by a push alarm, vibration, sound, etc.

In addition, in the formulating mode, when the temperature of the bottle drops below the formulating temperature and then reaches the formulating temperature again, the controller 820 may prevent an alarm from being raised during a grace period in order to prevent inconvenience due to repeated alarms.

Meanwhile, the controller 820 may drive the heater 830 to operate in the formulating mode when it is determined that formula preparation for milk has been completed by the inclination sensor 813, the weight measurement sensor 815, and the lid recognition sensor 817.

In addition, in the formulating mode, the controller 820 may control the ultraviolet lamp 850 to emit UV light to sterilize formula milk.

In this case, the controller 820 may drive the ultraviolet lamp 850 while the heater 830 is being driven, and may stop the operation of the ultraviolet lamp 850 when formula preparation for milk is completed.

The formulating mode may be switched to the feeding mode when a user presses a button provided on the base after preparing milk or inputs a mode change command to a portable device. In general, when preparing milk, the user shakes a bottle so that powdered milk is easily dissolved in water. This motion causes large changes in the inclination, acceleration, and/or angular velocity of the bottle. Therefore, when it is determined that a value measured by the inclination sensor in the formulating mode is equal to or greater than a threshold value, the controller 820 may determine that the user is preparing milk and automatically switch to the feeding mode.

On the other hand, when it is determined that a bottle is tilted at an angle equal to or wider than a preset angle for a longer time than a preset time, the controller 820 may determine that bottle-feeding has started. In this case, when the temperature of the bottle is equal to or higher than a preset temperature, the controller 820 may operate the motor 860 to provide a user with an alarm about the danger of burns by generating a vibration. For example, when the temperature of 38° C. or higher is measured 10 times or more in the formulating mode or the feeding mode, the controller 820 may determine that the bottle-feeding has started and may generate a vibration when the inclination of the bottle is maintained at a value of −1.2 g or more and at a value of an x or y axis from the reference (i.e., the gravity direction is set as a reference value of 0) for five seconds or more. Furthermore, when the inclination of the bottle is less than/equal to −0.5 g or equal to/more than −9 g from the reference in the feeding mode, the controller 820 may operate the motor 860 to generate a vibration, thereby inducing the user to feed milk at an appropriate angle.

Meanwhile, when the inclination sensor 813 is embodied as the gyro sensor, the controller 820 may calculate the inclination of a bottle based on an angular velocity measured by the gyro sensor. When the inclination sensor 813 is embodied as the acceleration sensor, the controller 820 may calculate the inclination of the bottle based on an acceleration measured by the acceleration sensor. The controller 820 may determine whether bottle-feeding has started or ended based on information on the inclination of the bottle obtained by the inclination sensor 813.

Specifically, the controller 820 may determine that feeding has started when it is determined that a bottle is tilted at an angle equal to or wider than a preset angle for a time equal to or longer than a preset time, and may determine that the feeding has ended when it is determined that the bottle is tilted at an angle narrower than or equal to the preset angle after a preset duration. In this case, when it is determined that the inclination of the bottle is outside a preset range for the preset duration, the controller 820 may control the heater 830 to maintain the temperature of the bottle at a preset temperature.

For example, when the inclination of a bottle is maintained at a value of −1.2 g or more and at a value of an x or y axis from the reference for five seconds or more, the controller 820 may determine that the condition for recording the start of feeding has been satisfied. In addition, when the controller 820 senses a feeding activity being performed for one minute or longer after the inclination of the bottle has reached an angle that satisfies the condition for recording the start of feeding, it may later regard the time point at which the condition for recording the start of feeding began not to be met as the time point at which the feeding ended. Furthermore, after switching to the feeding mode, when the duration of the feeding activity (time during which an inclination of −1.2 g or more is maintained, including when an inclination goes back to 0 g) is shorter than one minute, the controller 820 may determine that a user has temporarily stopped the feeding and may drive the heater 830 until the end of the feeding to maintain the feeding temperature for up to two hours. After two hours, the controller 820 may stop the operation of the heater 830 because the milk may spoil. In this case, a pop-up message recommending to prepare milk over again may be displayed on the portable terminal.

Meanwhile, the controller 820 may determine whether the formulating mode has started or ended based on the weight measurement sensor 815 and the lid recognition sensor 817.

For example, the controller 820 may determine that the formulating mode has started when a bottle is empty and a lid is not engaged, and may determine that the formulating mode has ended when the bottle is filled with liquid and the lid is engaged.

In this case, the controller 820 may control the heater 830 so that the temperature of the bottle reaches the formulating temperature.

Whether a bottle is empty or contains liquid may be determined based on the measurement by the weight measurement sensor 815.

In addition, the controller 820 may determine that the feeding mode has started when a bottle is filled with liquid and a lid is not engaged, and may determine that the feeding mode has ended when the weight of the liquid in the bottle is reduced and the lid is re-mounted thereon. In this case, the controller 820 may calculate the amount of fed milk by calculating how much the weight of the liquid in the bottle is reduced.

The heater 830 may heat liquid contained in a bottle. To this end, the heater 830 may be provided in a plate shape on the upper side of the base.

The status indicator light 840 may indicate information on bottle-feeding or preparation for formula milk.

The ultraviolet lamp 850 may emit UV light to sterilize a bottle or liquid in the bottle. The ultraviolet lamp 850 may be a UV-C LED. In this case, the ultraviolet lamp 850 may emit light in a vertical direction so that the light is not emitted to the outside of the bottle and may emit the light only at an angle within a certain range.

In this case, the ultraviolet lamp 850 may perform sterilization only when the bottle is filled with contents. In addition, the ultraviolet lamp 850 may emit UV light only when the weight of the bottle measured by the weight measurement sensor 815 is equal to or greater than a threshold value.

On the other hand, the ultraviolet lamp 850 may emit UV light only when the bottle is placed in a vertical direction of the ground. For example, the ultraviolet lamp 850 may emit UV light only when the inclination of the bottle measured by the inclination sensor 813 is in error by less than or equal to a threshold value with respect to the vertical direction of the ground.

In addition, the ultraviolet lamp 850 may emit UV light only when the lid recognition sensor 817 determines that a lid is coupled to a bottle in order to protect a user of the smart bottle from UV rays.

Furthermore, the ultraviolet lamp 850 may not emit UV rays in the formulating mode or the feeding mode.

As described above, the operation of the ultraviolet lamp 850 to emit or not emit UV rays may be performed under the control of the controller 820.

The motor 860 may generate a vibration to provide information on the inclination of a bottle, etc. For example, when it is determined that the bottle is inclined at an angle equal to or wider than a preset angle for a longer time than a preset time, the motor 860 may be driven by the controller 820 when the temperature of the bottle is equal to or higher than a preset temperature.

The communication unit 870 may transmit information on bottle-feeding such as information on a temperature measured by the temperature sensor 811, an inclination measured by the inclination sensor 813, etc., information on the remaining amount of the battery 880, and so on to an external device such as a repeater and a portal terminal.

In addition, the communication unit 870 may receive information on setting the feeding temperature, information on setting the formulating temperature, information on mode change, etc. from the external device.

The communication unit 870 may include an NFC chip, an NFC antenna, a Bluetooth module, a Wi-Fi module, a 5G communication module, and the like. For example, the communication unit 870 may communicate with a terminal by a Bluetooth 4.0 communication.

The battery 880 may supply power to the smart bottle. When the controller 820 checks the remaining amount of the battery 880 and determines that it is not possible to heat the bottle to the formulating temperature and/or the feeding temperature, it may provide an alarm about the shortage of the remaining amount of the battery 880 by means of the status indicator light 840 and/or the motor 860 without driving the heater 830. In that case as well, the portable terminal may also raise an alarm about the low remaining amount of the battery 880 by a push alarm, vibration, sound, etc.

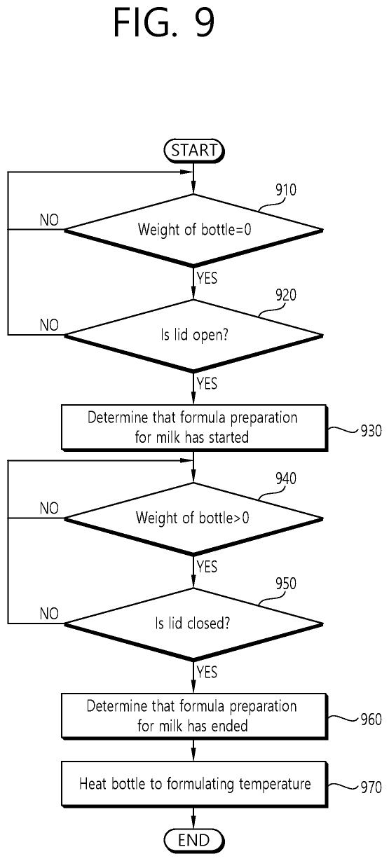

FIG. 9 shows a method of determining whether formula preparation for milk has started or ended according to an embodiment of the present disclosure. The operations described below may be performed by a base of a smart bottle.

Referring to FIG. 9, the base may determine whether the bottle is empty by measuring the weight of liquid contained in the bottle at S910.

When the bottle is empty, the base may determine whether a lid of the bottle is open to determine whether the lid has been opened by a user at S920. In this case, information obtained by a lid recognition sensor provided in the bottle may be transmitted to the base so that whether the lid is open may be determined.

When the lid is open while the bottle is empty, the smart bottle may determine that formula preparation for milk has started at S930.

When there is liquid in the bottle or the lid is kept closed, the base may determine that the formula preparation for milk has not yet started and may continuously weigh the bottle and check that the lid has been opened.

When it is determined that the formula preparation for milk has started, the base may determine whether the bottle is being filled with liquid by measuring the weight of the bottle at S940.

When the liquid is contained in the bottle, the base may determine whether the bottle lid is closed to determine whether the lid has been closed by the user at S950.

When the bottle lid is closed while the liquid is contained in the bottle, the base may determine that the formula preparation for milk has been completed at S960 and may heat the bottle to a formulating temperature at S970.

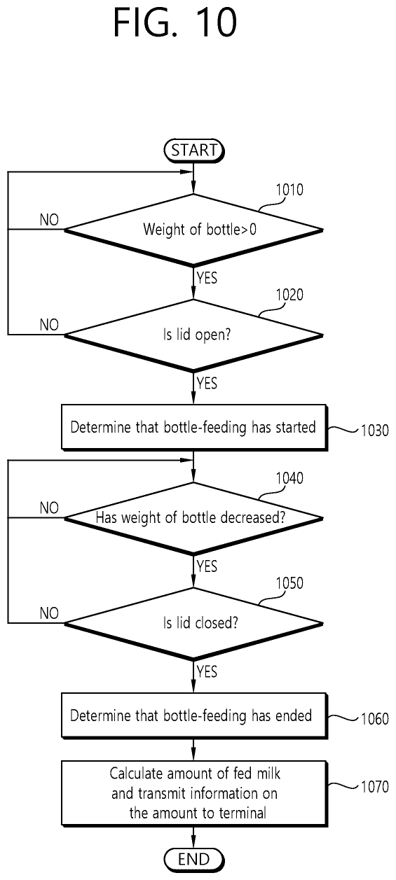

FIG. 10 shows a method of determining whether bottle-feeding has started or ended according to an embodiment of the present disclosure. The operations described below may be performed by a base of a smart bottle.

Referring to FIG. 10, the base may determine whether liquid is contained in the bottle by measuring the weight of liquid contained in the bottle at S1010.

When liquid is contained in the bottle, the base may determine whether a lid of the bottle is open to determine whether the lid has been opened by a user at S1020. In this case, information obtained by a lid recognition sensor provided in the bottle may be transmitted to the base so that whether the lid is open may be determined.

When the bottle lid is open while liquid is contained in the bottle, the base may determine that bottle-feeding has started at S1030.

When the bottle is empty or the bottle lid is kept closed, the base may determine that the bottle-feeding has not yet started and may continuously weigh the bottle and check that the lid has been opened.

When it is determined that the bottle-feeding has started, the base may determine whether the weight of the liquid in the bottle is decreasing by measuring the weight of the bottle at S1040.

When the weight of the liquid contained in the bottle is reduced, the base may determine whether the bottle lid is closed to determine whether the lid has been closed by the user at S1050.

When the weight of the liquid contained in the bottle is reduced and the bottle lid is closed, the base may determine that the bottle-feeding has been completed at S1060, and may calculate the amount of fed milk based on how much the weight of the liquid in the bottle is reduced and transmit information on the calculated amount to a terminal at S1070.

As described above, there is no limitation in applying the features and the methods in the above-mentioned embodiments described in relation to the smart bottle and the method of controlling the same according to the present disclosure, and all or part of the embodiments may be selectively combined for various modifications of the embodiments.

What is claimed is:

1. An apparatus comprising:
a bottle for containing liquid; and
a base disposed under the bottle,
wherein the base includes:
an elastic body that is elastically deformed by the weight of the bottle and the base;
a weight measurement sensor disposed above the elastic body and measuring the weight of the bottle by measuring a pressure that the elastic body transfers in a vertical direction from the ground as a reaction to gravity; and
a controller that measures the amount of fed milk based on the change in the weight measured by the weight measurement sensor.

2. The apparatus of claim 1 further comprising a lid,
wherein the bottle includes a lid recognition sensor for determining whether the lid is engaged.

3. The apparatus of claim 2,
wherein the controller determines that formula preparation for milk has started when the lid is not engaged while the bottle is empty and determines that the formula preparation for milk has ended when the lid is engaged while the bottle is filled with contents.

4. The apparatus of claim 2,
wherein the controller determines that bottle-feeding has started when the lid is not engaged while the bottle is filled with contents and determines that the bottle-feeding has ended when the weight of the bottle is reduced and the lid is engaged.

5. The apparatus of claim 4,
wherein the controller measures the amount of fed milk based on the change in the weight of the bottle.

6. The apparatus of claim 1,
wherein the base further includes an ultraviolet (UV-C) lamp for sterilizing the contents in the bottle by emitting ultraviolet light toward the bottle.

7. The apparatus of claim 6,
wherein the ultraviolet lamp emits UV light when the weight of the bottle is equal to or greater than a threshold value.

8. The apparatus of claim 6,
wherein the base further includes an inclination sensor for measuring the inclination of the bottle, and the ultraviolet lamp emits UV light when the inclination of the bottle is in error by less than or equal to a threshold value with respect to a vertical direction of the ground.

9. The apparatus of claim 8,
wherein the inclination sensor includes at least one of a gyro sensor or an acceleration sensor, and the controller calculates a value of inclination based on information on an angular velocity measured by the gyro sensor or based on information on an acceleration measured by the acceleration sensor.

10. The apparatus of claim 1,
wherein the weight measurement sensor includes at least one of a beam-type load cell, a three-wire load cell, and a columnar load cell.

11. A method of operating an apparatus configured to be engageable on one side of a bottle, comprising:
determining whether the bottle is empty by measuring the weight of liquid contained in the bottle;
determining whether a lid of the bottle is open; and
determining that preparation for formula milk has started when the lid is open while the bottle is empty.

12. The method of claim 11 further comprising:
determining whether the bottle is being filled with liquid by measuring the weight of the bottle when it is determined that the preparation for formula milk has started;
determining whether the lid is closed;
determining that the preparation for formula milk has ended when the lid is closed while the liquid is contained in the bottle; and
heating the bottle to a formulating temperature.

13. The method of claim 12 further comprising:
determining whether liquid is contained in the bottle by measuring the weight of liquid contained in the bottle;
determining whether the lid is open; and
determining that bottle-feeding has started when the lid is open while the liquid is contained in the bottle.

14. The method of claim 13 further comprising:
determining whether the weight of the liquid in the bottle is decreasing by measuring the weight of the bottle when it is determined that the bottle-feeding has started;
determining whether the bottle lid is closed;
determining that the bottle-feeding has ended when the weight of the liquid is reduced and the bottle lid is closed; and
calculating the amount of fed milk and transmitting information on the calculated amount to a terminal.
